(19) United States
(12) Patent Application Publication
(10) Pub. No.: US 2019/0298275 A1
(43) Pub. Date: Oct. 3, 2019

(54) X-RAY IMAGE DIAGNOSTIC APPARATUS

(71) Applicant: Shimadzu Corporation, Kyoto-shi (JP)

(72) Inventors: Keita OKUTANI, Kyoto-shi (JP); Masahiro Tanaka, Kyoto-shi (JP)

(73) Assignee: Shimadzu Corporation, Kyoto-shi (JP)

(21) Appl. No.: 16/259,607

(22) Filed: Jan. 28, 2019

(30) Foreign Application Priority Data
Mar. 30, 2018 (JP) 2018-66679

(51) Int. Cl. A61B 6/04; A61B 6/00

(52) U.S. Cl. CPC A61B 6/0407; A61B 6/4447; A61B 6/487; A61B 6/102; A61B 6/461; G06T 2207/10116; A61B 6/481; A61B 6/547

(57) ABSTRACT

An X-ray image diagnostic apparatus, comprises: a table on which a subject is placed on the front surface thereof; an X-ray tube that is disposed beneath the table and emits X-rays to the subject; a detector that is disposed above the table so as to face the X-ray tube and detects the X-rays emitted from the X-ray source and penetrating the subject placed on the table; an X-ray tube moving mechanism that moves the X-ray tube in a direction approaching or away from the table; a detector moving mechanism that moves the detector in a direction approaching or away from the table; and a control unit that controls a distance between the X-ray tube and the detector by controlling action of the X-ray tube moving mechanism and the detector moving mechanism.

### CROSS REFERENCE TO RELATED APPLICATIONS

This application relates to, and claims priority from JP 2018-066679 filed Mar. 30, 2018, the entire contents of which are incorporated herein by reference.

### FIGURE SELECTED FOR PUBLICATION

FIG. 1.

### BACKGROUND OF THE INVENTION

### Field of the Invention

The present invention relates to an X-ray image diagnostic apparatus.

### Description of the Related Art

Among X-ray image diagnostic apparatuses, there is an apparatus called a proximity fluoroscopic apparatus that is used when a user, such as medical doctor, takes an X-ray image of a subject, such as a patient, while the user operates the apparatuses in the proximity of the subject. Among these, a proximity fluoroscopic apparatus; including an X-ray tube, which is provided beneath a table, on which the patient is placed, and a detector, which is provided above the table and detects X-rays emitted from the X-ray tube; is called an under-table (X-ray) tube proximity operation fluoroscopic imaging apparatus (for example, refer to JP2001-000428A).

With respect to the conventional under-table tube proximity operation fluoroscopic imaging apparatus, whereas the detector is movable in a direction approaching or moving away from the table, the X-ray tube is not movable in a direction approaching or moving away from the table.

In addition, in order to suppress the influence of scattered rays, a scattered ray removal grid is mounted on the detector. As to the proximity fluoroscopic apparatus, since a distance (source image distance: SID) between the X-ray tube and the detector is short, a convergence grid is used in many cases. In the convergence grid, depending on the convergence distance, a transmission portion and an absorption portion are arranged so as to be inclined at a predetermined angle in the thickness direction of the grid.

For this reason, when the SID become mismatched to the convergence distance of the grid, some of the X-rays emitted from the X-ray tube are absorbed by the absorption portion of the grid, and a uniform image cannot be obtained. Accordingly, it is important to select a grid having an appropriate convergence distance matching to the SID.

For example, in an under-table tube proximity fluoroscopic apparatus, the detector may be moved in a direction approaching or away from the subject (or the table) depending on an imaging part (position of a region of interest) or the thickness (body thickness) of the subject.

However, as described above, in the conventional under-table tube proximity operation fluoroscopic imaging apparatus, the X-ray tube is not movable in a direction approaching or away from the table. Therefore, when the detector is moved in a direction approaching or away from the table, the SID dynamically changes and as a result, become mismatched to the convergence distance of the grid.

In the above case, each time the SID changes, the grid must be changed to another grid having the convergence distance suitable for the SID. However, such a task is a heavy burden on the user.

### RELATED PRIOR ART DOCUMENT

### Patent Document

Patent Document: JP2001-000428(A)

### ASPECTS AND SUMMARY OF THE INVENTION

An object of the present invention is to provide an X-ray image diagnostic apparatus capable of imaging a subject with X-rays in accordance with a purpose without increasing the burden on a user.

The X-ray image diagnostic apparatus according to an aspect of the present invention comprises: a table that has a front surface and a back surface, and a subject is placed on the front surface thereof; an X-ray source that is disposed on the beneath side of the table and emits X-rays to the subject; a detector that is disposed on the front surface (above) side of the table so as to face the X-ray source and that detects the X-rays emitted from the X-ray source and are penetrating the subject placed on the table; a first moving mechanism that moves the X-ray source in a direction approaching or away from the table; a second moving mechanism that moves the detector in a direction approaching or away from the table; and a control unit that controls a distance between the X-ray source and the detector by controlling actions of the first moving mechanism and the second moving mechanism.

In the X-ray image diagnostic apparatus according to the aspect of the present invention, as described above, the control unit individually controls the movement of the X-ray source by the first moving mechanism and the movement of the detector by the second moving mechanism. Therefore, the operability of the X-ray image diagnostic apparatus is high, so that the positional relationship between the X-ray source and the detector can be set with more freedom.

In the X-ray image diagnostic apparatus according to the aspect of the present invention, the control unit can control the action of the second moving mechanism in response to an input from an operator and control the action of the first moving mechanism such that the X-ray source is moved interlockingly with movement of the detector controlled by the second moving mechanism. By the configuration described above, for example, the X-ray imaging of the subject can be more easily performed in accordance with the purpose without increasing the burden on the user.

In the X-ray image diagnostic apparatus in which the X-ray source is moved interlockingly with the movement of the detector, the control unit can control the action of the first moving mechanism in order to keep a distance (SID) between the X-ray source and the detector being constant in a movable range of the X-ray source. By the configuration described above, the X-ray imaging of the subject can be performed in the optimal state for the purpose of keeping the SID being constant.

In this case, the X-ray image diagnostic apparatus further comprise: a mounting unit that enables mounting a grid for removing a scattered ray on the detector; and a grid detection unit that detects the kind of the grid and determines whether such a grid is mounted onto the mounting unit or not, and the control unit can determine the away distance in accordance with the kind of the grid detected by the grid detection unit. According to the configuration described above, the convergence distance of the grid and the SID always match with each other. Therefore, most of the X-rays emitted from the X-ray source transmit a transmission portion of the grid, so that a uniform (homogeneous) X-ray image of the subject can be obtained. As a result, the accurate diagnosis can be achieved based on the obtained image.

In the X-ray image diagnostic apparatus in which the X-ray source is moved in conjunction with the movement of the detector, it is preferable that the control unit can control the action of the first moving mechanism so that an enlargement ratio of an X-ray image of the subject is kept being constant. By the configuration described above, the X-ray imaging of the subject can be performed in the optimal state for the purpose of keeping the enlargement ratio of the image being constant.

The X-ray image diagnostic apparatus according to the aspect of the present invention further comprises a lifting mechanism that moves up and down the table in a horizontal state together with the first moving mechanism and the second moving mechanism, and the control unit can control the action of the first moving mechanism in order to move the X-ray source interlockingly with moving up and down the table in the horizontal state by the lifting mechanism. By the configuration described above, for example, the table can be brought closer to the installation surface on which the X-ray image diagnostic apparatus is installed. As a result, such a configuration facilitates the subject to get on and off the table.

In this case, the control unit can control the action of the first moving mechanism in order to move the X-ray source in a direction approaching the table when a distance between an installation surface on which the X-ray image diagnostic apparatus is installed and the X-ray source is a predetermined value. By the configuration described above, the X-ray source can be prevented from colliding with the installation surface to be damaged and the table can be set closer to the installation surface (i.e., the table can be lowered closer to a lower position).

### Effect of the Invention

According to the aspect of the present invention, for example, the X-ray imaging of the subject can be performed in accordance with the purpose without increasing the burden on the user.

The above and other aspects, features and advantages of the present invention will become apparent from the following description read in conjunction with the accompanying drawings, in which like reference numerals designate the same elements.

### BRIEF DESCRIPTION OF THE DRAWINGS

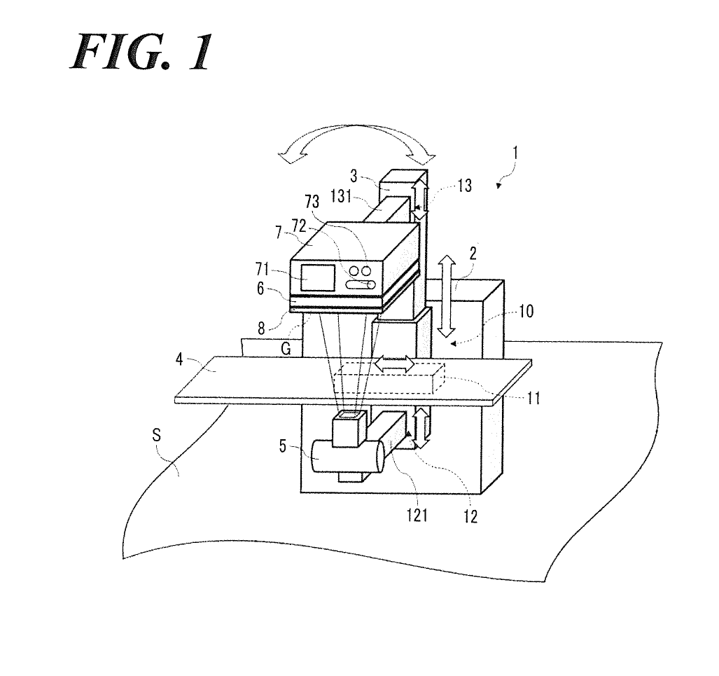

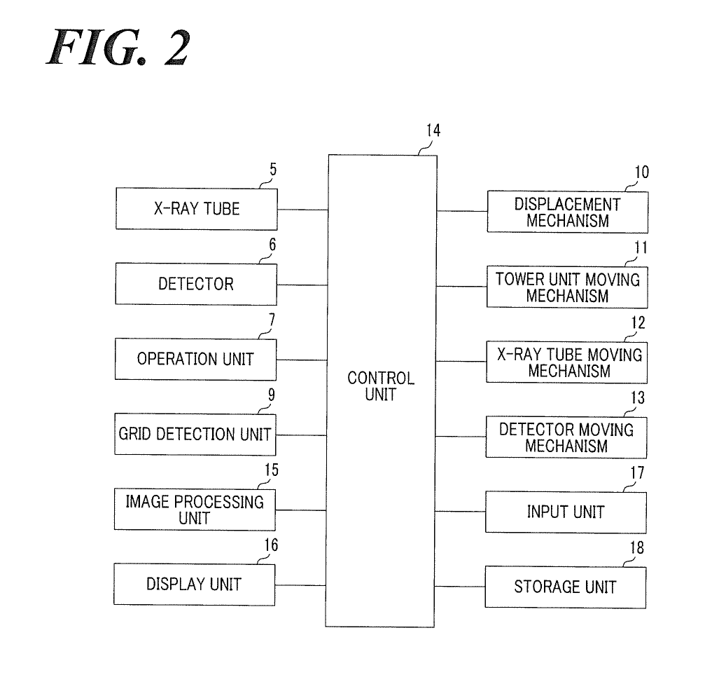

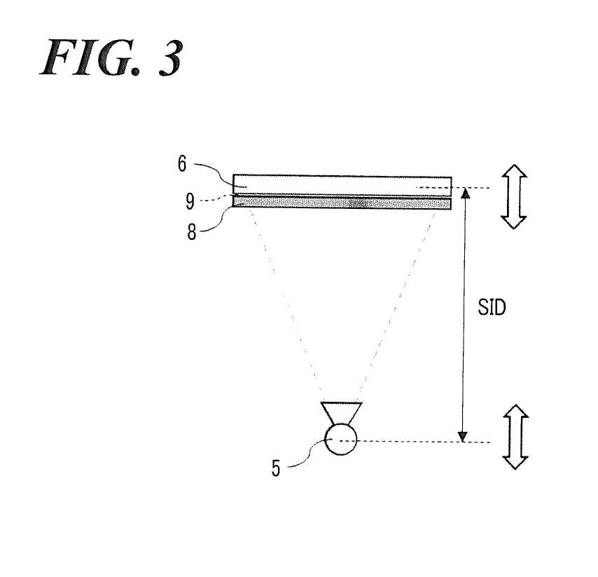

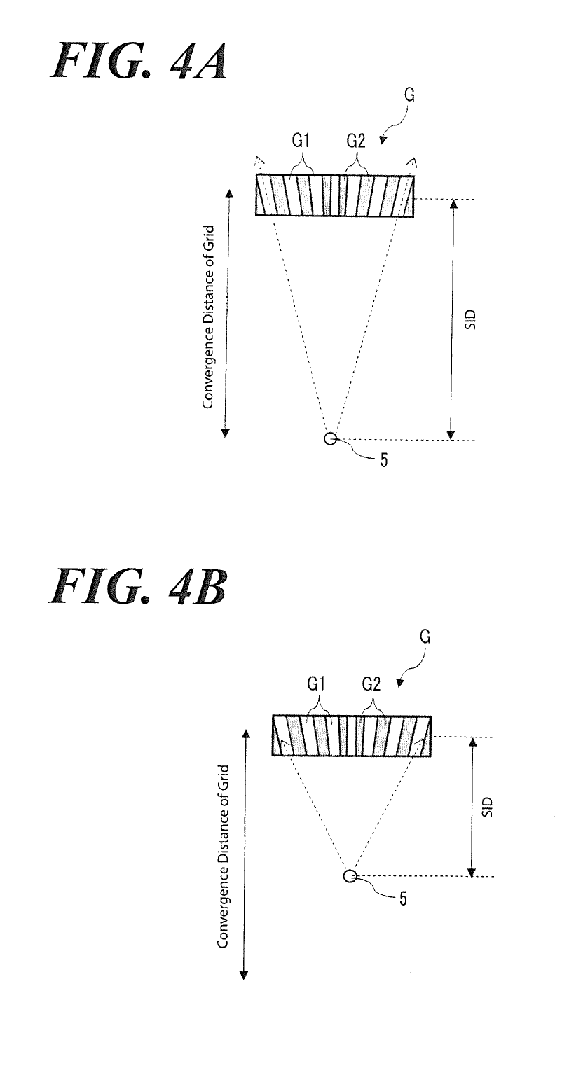

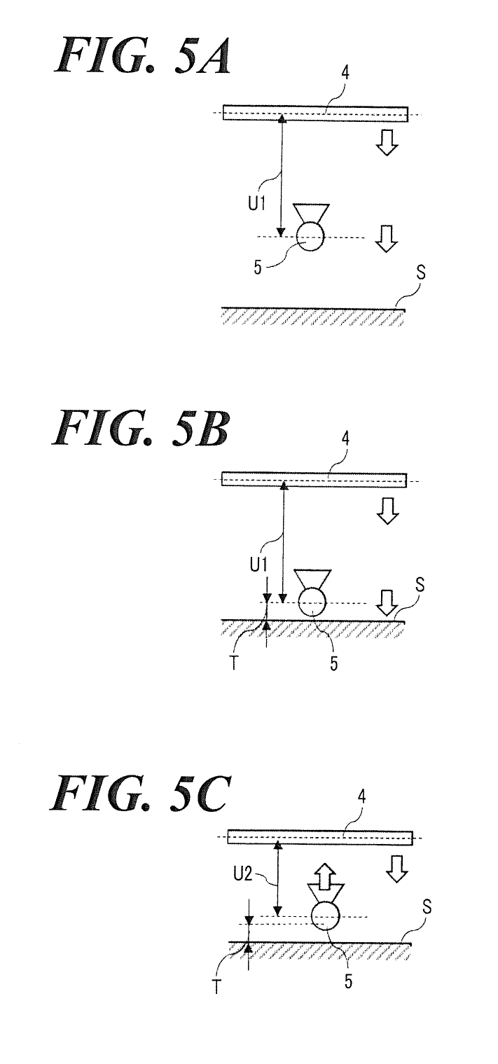

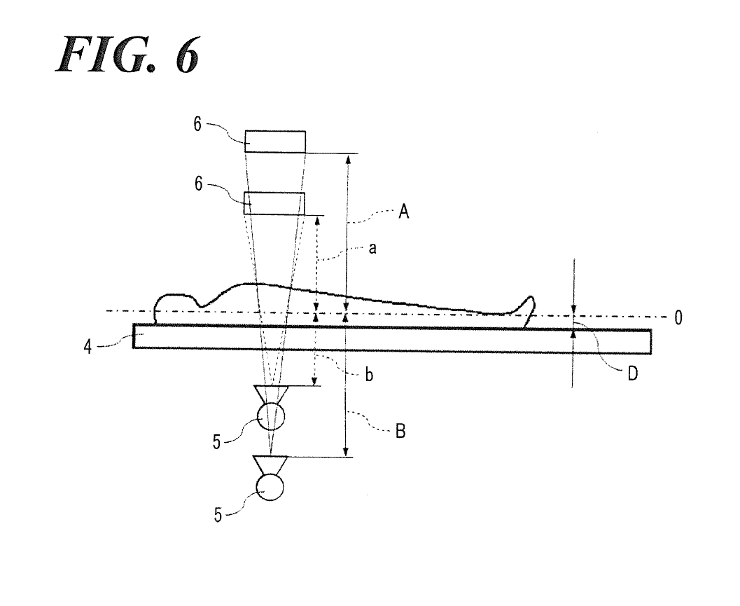

FIG. 1 is a schematic diagram showing the overall configuration of an X-ray image diagnostic apparatus according to an embodiment of the present invention.

FIG. 2 is a block diagram of the X-ray image diagnostic apparatus shown in FIG. 1.

FIG. 3 is a diagram showing the positional relationship between an X-ray tube and a detector.

FIGS. 4A and 4B are diagrams illustrating the relationship between a grid and a SID.

FIGS. 5A, 5B, and 5C are diagrams showing the positional relationship between an X-ray tube and a table.

FIG. 6 is a diagram showing the positional relationship between an X-ray tube and a detector relative to a subject.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ or ‘connect’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

Hereinafter, an X-ray image diagnostic apparatus of the present invention will be described in detail based on a preferred embodiment shown in the accompanying diagrams.

FIG. 1 is a schematic diagram showing the overall configuration of an X-ray image diagnostic apparatus according to an embodiment of the present invention, FIG. 2 is a block diagram of the X-ray image diagnostic apparatus shown in FIG. 1, FIG. 3 is a diagram showing the positional relationship between an X-ray tube and a detector, FIGS. 4A and 4B are diagrams illustrating the relationship between a grid and a SID, FIGS. 5A, 5B, and 5C are diagrams showing the positional relationship between an X-ray tube and a table, and FIG. 6 is a diagram showing the positional relationship between an X-ray tube and a detector to a subject.

In the respective diagrams, in order to make the features easy to understand, a characteristic portion may be shown in an enlarged scale for convenience, and the dimensional ratios and the like of respective components may be different from the actual ones. Materials, dimensions, and the like exemplified below are only examples, and the present invention is not limited thereto and can be appropriately changed within a range not changing the gist of the present invention.

An X-ray image diagnostic apparatus 1 shown in FIG. 1 is an apparatus that takes an X-ray image to image the inside of a subject by emitting X-rays from the outside of the subject, such as a human body.

The X-ray image diagnostic apparatus 1 has a support unit (leg unit) 2, a main frame unit that is provided so as to be displaced (moving up and down and rotating) with respect to the support unit 2 and holds a table 4 (not shown), a tower unit 3 provided so as to be movable (slidable) with respect to the main frame unit, an X-ray tube (X-ray source) 5 and a detector 6 that are provided so as to be movable with respect to the tower unit 3, and an operation unit 7 fixed to the detector 6.

In the state shown in FIG. 1, the X-ray tube 5 is disposed below the table 4 (on the back-surface side of the table 4), and the detector 6 is disposed above the table 4 (on the front surface side of the table 4) so as to face the X-ray tube 5. That is, the X-ray image diagnostic apparatus 1 is a so-called under-table tube proximity fluoroscopic apparatus. According to the X-ray image diagnostic apparatus 1, the exposure of scattered rays against a user (such as a medical doctor) can be suppressed, so that the safety becomes higher.

The support unit 2 is installed on an installation surface S of an imaging room where the X-ray image diagnostic apparatus 1 is installed. The support unit 2 has a function of supporting each unit of the X-ray image diagnostic apparatus 1. A main frame unit that holds the table 4 is attached to the support unit 2. The table 4 has a front surface and a back surface, and a subject is placed on the front surface while lying down.

The tower unit 3 is attached to the main frame unit. The X-ray tube 5 is attached to a lower end portion of the tower unit 3, and the detector 6 is attached to an upper end portion of the tower unit 3.

The X-ray tube 5 is connected to a high voltage (power) generation unit (not shown in FIG.). A high voltage is applied to the X-ray tube 5 to generate X-rays, and the X-rays are emitted to the subject. On the other hand, the detector 6 includes an X-ray conversion unit (a plurality of semiconductor X-ray detection elements arranged in a matrix) thereinside and detects X-rays that are emitted from the X-ray tube 5 and penetrates the subject placed on the table 4.

The operation unit 7 is fixed to an upper portion of the detector 6. The operation unit 7 is a unit used when the user (such as a doctor) performs an operation to move the detector 6, and includes a monitor 71, an operation handle 72, and a switch 73, and the like.

As shown in FIG. 3, a mounting unit 8 that enables mounting the scattered ray removal grid G on the detector 6 and a grid detection unit 9 capable of detecting the presence or absence of mounting of the grid G onto the mounting unit 8 and the kind of the grid G are provided below the detector 6.

The mounting unit 8 includes, for example, a pair of rails for guiding the grid G by inserting two opposite edge portions (sides) of the grid G therebetween. The grid G has a characteristic (convergence) that allows only X-rays from the X-ray tube 5, for which the distance from the detector 6 (SID) is set to provide a predetermined value, to pass therethrough and does not allow scattered rays due to the subject to pass therethrough. Plural kinds of the grid G providing different convergence distances are available.

The kind of the grid G (that is, the convergence distance) is detected by the grid detection unit 9. For example, the grid detection unit 9 can be formed by a photosensor that reads an optical pattern indicating the kind of the grid G or a magnetic sensor that reads magnetic data indicating the kind of the grid G. The optical pattern and the magnetic data are provided at the end portion of the grid G.

In addition, the X-ray image diagnostic apparatus 1 has a displacement mechanism 10 for supporting the main frame unit, which holds the table 4, so as to be movable up and down and rotatable with respect to the support unit 2, a tower unit moving mechanism 11 for supporting the tower unit 3 so as to be slidable with respect to the main frame unit, and an X-ray tube moving mechanism (first moving mechanism) 12 and a detector moving mechanism (second moving mechanism) 13 for supporting the X-ray tube 5 and the detector 6 so as to be movable with respect to the tower unit 3, respectively.

The displacement mechanism 10 moves (moves up and down) the table 4 (main frame unit) in the horizontal state in the vertical direction together with the X-ray tube 5 and the detector 6, and rotates the table 4, the X-ray tube 5, and the detector 6 with respect to the support unit 2.

The tower unit moving mechanism 11 slides the tower unit 3 in the longitudinal direction of the main frame unit (table 4). As a result, the X-ray tube 5 and the detector 6 slide in the longitudinal direction with respect to the table 4.

The X-ray tube moving mechanism 12 includes an arm 121 to which the X-ray tube 5 is fixed and a motor (not shown) for moving the arm 121 along the longitudinal direction of the tower unit 3. By rotating the motor to move the arm 121, the X-ray tube moving mechanism 12 moves the X-ray tube 5 in a direction approaching or away from the table 4 (a direction perpendicular to the table 4).

The detector moving mechanism 13 includes an arm 131 to which the detector 6 is fixed and a motor (not shown) for moving the arm 131 along the longitudinal direction of the tower unit 3. By rotating the motor to move the arm 131, the X detector moving mechanism 13 moves the detector 6 in a direction approaching or away from the table 4 (a direction perpendicular to the table 4).

The detector 6 is movable (slidable) along the longitudinal direction of the arm 131. Therefore, when the detector 6 is retracted to the back position on the support unit 2 side, the detector 6 is not obstructive during an operation in which the subject gets on and off the table 4.

In the configuration described above, the displacement mechanism 10 can collectively move (move up and down) the main frame unit that holds the table 4, the tower unit 3 and the tower unit moving mechanism 11 provided in the main frame unit, and the X-ray tube 5, the X-ray tube moving mechanism 12, the detector 6, and the detector moving mechanism 13 provided in the tower unit 3 in the vertical direction with respect to the support unit 2 and collectively rotate these with respect to the support unit 2.

The X-ray image diagnostic apparatus 1 has a control unit 14 connected to the X-ray tube 5, the detector 6, the operation unit 7, the grid detection unit 9, the displacement mechanism 10, the tower unit moving mechanism 11, the X-ray tube moving mechanism 12, and the detector moving mechanism 13.

The control unit 14 is a computer including a central processing unit (CPU), a read only memory (ROM), a random access memory (RAM), and the like. The control unit 14 controls the action of the X-ray tube moving mechanism 12 and the detector moving mechanism 13 by executing a predetermined control program with the CPU, thereby controlling the distance between the X-ray tube 5 and the detector 6 and the operation of each unit of the X-ray image diagnostic apparatus 1.

The control unit 14 can individually control the movement of the X-ray tube 5 by the X-ray tube moving mechanism 12 and the movement of the detector 6 by the detector moving mechanism 13 without the movements being controlled in conjunction with each other. In the present embodiment, however, the operation of the X-ray tube moving mechanism 12 is controlled so that the X-ray tube 5 is moved interlockingly with the movement of the detector 6 by the detector moving mechanism 13.

Therefore, by simply operating the operation unit 7, the movement of the detector 6 and the movement of the X-ray tube 5 interlockingly therewith can be performed under the control of the control unit 14. Therefore, the operability of the X-ray image diagnostic apparatus 1 is higher, so that when the positional relationship between the X-ray tube 5 and the detector 6 is set in advance, an X-ray imaging of the subject in accordance with the purpose can be performed.

The X-ray image diagnostic apparatus 1 has an image processing unit 15, a display unit 16, an input unit 17, and a storage unit 18 that are respectively connected to the control unit 14.

The image processing unit 15 is, for example, a computer including a CPU or a graphics processing unit (GPU). The image processing unit 15 executes a predetermined image processing program. The image processing unit 15 can be configured integrally with the control unit 14 by executing the image processing program using the same hardware (CPU) as for the control unit 14.

The display unit 16 can be, for example, a liquid crystal display or an organic EL display. On the display unit 16, the image generated by the image processing unit 15 is displayed under the control of the control unit 14.

The input unit 17 can be, for example, a keyboard and a mouse, a touch panel, or other controllers. The user (such as a medical doctor) performs various input operations on the input unit 17, and the control unit 14 receives the input operations.

The storage unit 18 can be a storage device such as a hard disk drive, for example. The storage unit 18 stores a control program and an image processing program and stores the data of the image, imaging conditions, and a variety of setting values.

Examples of the setting values include a value of SID corresponding to the kind of the grid G (convergence distance), a ratio of the distance between the centerline in the thickness direction of the subject and the X-ray tube 5 and the distance between the centerline in the thickness direction of the subject and the detector 6, and the like.

The convergence distance of the grid G and the SID need not completely the same. For example, when the convergence distance of the grid G is 100 cm, the SID is set in the range of 80 cm to 120 cm.

Next, the operation (usage) of the X-ray image diagnostic apparatus 1 when the SID is kept constant will be described.

First, a user (such as a medical doctor who is an operator) mounts the grid G to the mounting unit 8. The grid detection unit 9 detects that the grid G has been mounted on the mounting unit 8, and identifies the kind of the grid G. The identification (detection) signal is transmitted to the control unit 14, and then the control unit 14 determines the SID corresponding to the kind of the grid G based on the setting value stored in the storage unit 18.

Then, the user operates the operation handle 72 of the operation unit 7 vertically and horizontally so that the table 4 and the detector 6 are in a desired relative position. Through the above operation, the control unit 14 controls the action of the detector moving mechanism 13 so that the detector 6 is moved in a direction approaching or away from the table 4 and controls the action of the tower unit moving mechanism 11 so that the tower unit 3 slides in the longitudinal direction of the table 4 (main frame unit). Thereafter, the user presses the predetermined switch 73 of the operation unit 7 to start the emission of X-rays from the X-ray tube 5 to a region of interest of the subject (region to be imaged for diagnosis).

The X-rays emitted from the X-ray tube 5 penetrate the subject placed on the table 4 and are detected by the detector 6. The signal detected by the detector 6 is transmitted to the image processing unit 15, and an image (X-ray image) is formed. The formed image is displayed on the display unit 16 through the control unit 14.

Thereafter, in order to acquire an image of a different region of interest of the subject, the user operates the operation handle 72 of the operation unit 7 vertically and horizontally again to control the relative position between the table 4 and the detector 6. In this case, the control unit 14 controls the action of the detector moving mechanism 13 in response to the user's operation using the operation unit 7 (that is, the operator's input), and controls the action of the X-ray tube moving mechanism 12 so that the X-ray tube 5 is moved interlockingly with the movement of the detector 6 by the detector moving mechanism 13. Specifically, the control unit 14 controls the action of the X-ray tube moving mechanism 12 so that the SID between the X-ray tube 5 and the detector 6 is kept constant in the movable range of the X-ray tube 5.

In the present embodiment, the control unit 14 controls the action of the X-ray tube moving mechanism 12 so that the SID (i.e., the constant SID) determined according to the kind of the grid G mounted on the mounting unit 8 is maintained. Here, as shown in FIGS. 4A and 4B, the grid G includes a transmission portion G1 that X-rays transmit and an absorption portion G2 that absorbs X-rays, and the transmission portion G1 and the absorption portion G2 are arranged so as to incline at a predetermined angle in the thickness direction of the grid G.

In the above configuration, when the convergence distance of the grid G and the SID do not match with each other due to the change of the SID, some of the X-rays emitted from the X-ray tube 5 are absorbed by the absorption portion G2 (in particular, the outer absorption portion G2), so that a uniform (even) X-ray image of the subject, as shown in FIG. 4B.

In contrast, in the present embodiment, the SID determined according to the kind of the grid G is maintained, so that the convergence distance of the grid G and the SID always match with each other. Therefore, as shown in FIG. 4A, most of the X-rays emitted from the X-ray tube 5 can pass through the transmission portion G1 to provide a uniform X-ray image of the subject. As a result, an accurate diagnosis based on the obtained image can be performed.

In addition, the SID is kept constant, so that it is not necessary to replace the grid G with a grid having a different convergence distance. Therefore, the burden on the user does not increase.

Then, when the X-ray imaging of the subject ends, the user presses the predetermined switch 73 of the operation unit 7 to stop the emission of X-rays from the X-ray tube 5 to the subject. At this time, when the table 4 (main frame unit) is not in the horizontal state, the control unit 14 operates the displacement mechanism 10 to rotate the main frame unit with respect to the support unit 2 so that the table 4 is in the horizontal state. According to the above-described rotation of the main frame unit, the tower unit 3 also rotates, so that the longitudinal direction of the tower unit 3 coincides with the vertical direction.

The control unit 14 operates the X-ray tube moving mechanism 12 to move (lower) the X-ray tube 5 to the lowermost portion of the tower unit 3. At this point in time, the table 4 and the X-ray tube 5 are spaced away from each other by a distance U1 as shown in FIG. 5A.

Thereafter, in order to make it easier for the subject to get off the table 4, it is necessary to move (lower) the table 4 as vertically downward as possible. Therefore, in a case where the user presses the predetermined switch 73 of the operation unit 7, the control unit 14 operates the displacement mechanism 10 to move (lower) the table 4 (main frame unit) in the horizontal state downward in the vertical direction together with the X-ray tube 5. In this case, as shown in FIG. 5B, the X-ray tube 5 approaches quickest the installation surface S on which the X-ray image diagnostic apparatus 1 is placed.

In the present embodiment, the control unit 14 controls the action of the X-ray tube moving mechanism 12 so that the X-ray tube 5 is moved in conjunction with the vertical movement of the table 4 in the horizontal state by the displacement mechanism (lifting mechanism) 10. Specifically, when a distance T between the installation surface S and the X-ray tube 5 reaches a predetermined value as shown in FIG. 5B, the control unit 14 controls the action of the X-ray tube moving mechanism 12 so that the X-ray tube 5 is moved (raised) in a direction approaching the table 4 (upward vertically) as shown in FIG. 5C. The predetermined value of the distance T is not particularly limited, and preferably 10 cm or less, more preferably about in the range of 3 cm to 7 cm.

Through the configuration described above, the X-ray tube 5 can be prevented from colliding with the installation surface (floor surface) S to be damaged.

In addition, since an obstacle colliding with the installation surface S prior to the tower unit 3 disappears by moving the X-ray tube 5 upward vertically, the main frame unit that moves integrally with the tower unit 3 further can move downward in the vertical direction. Therefore, the table 4 held in the main frame unit can be brought closer to the installation surface S. As a result, the subject can easily get off the table 4. At this point in time, the table 4 and the X-ray tube 5 are spaced away from each other by a distance U2, which is shorter than the distance U1, as shown in FIG. 5C.

It goes without saying that such an operation can be performed when the subject is placed on the table 4.

In the present embodiment, whereas the grid G is used in the case of taking an X-ray image of the subject, using the X-ray image diagnostic apparatus 1, the grid G is not mandatory. In this case, the X-ray image diagnostic apparatus 1 is structured so that the user can set (determine) a constant SID by pressing the predetermined switch 73 of the operation unit 7 with the user's own discretion.

For example, in the case of performing imaging using a contrast medium (barium), the user checks the contrast medium in the image displayed on the display unit 16 while operating the operation unit 7 to move the detector 6 vertically and horizontally. Then, at a point in time at which the user can clearly recognize the contrast medium in the image, the user can set the SID to a constant value by pressing the predetermined switch 73 of the operation unit 7. According to the configuration described above, the movement of the contrast medium in the subject can be more accurately grasped.

In addition, when a relatively large SID is kept constant, an image of the subject can be taken with X-rays that is near parallel lights. In this case, an image with less distortion can be obtained.

Instead of keeping the distance (SID) between the X-ray tube 5 and the detector 6 constant, the control unit 14 can control the action of the X-ray tube moving mechanism 12 so that the enlargement ratio of the image of the subject by X-rays is kept constant. Also, in this case, the control unit 14 controls the action of the X-ray tube moving mechanism 12 so that the X-ray tube 5 is moved in the movable range of the X-ray tube 5.

According to the configuration described above, the control unit 14 keeps the ratio of the distance between the centerline O of the subject and the X-ray tube 5 and the distance between the centerline O of the subject and the detector 6 constant without taking the SID into consideration. Specifically, as shown in FIG. 6, the control unit 14 performs control such that a ratio of a distance a between the detector 6 and the centerline O and a distance b between the X-ray tube 5 and the centerline O is equal to a ratio of a distance A between the detector 6 and the centerline O and a distance B between the X-ray tube 5 and the centerline O.

For example, the user checks an observation (diagnosis) target in the image in the region of interest of the subject displayed on the display unit 16 while operating the operation unit 7 to move the detector 6 vertically and horizontally. Then, at a point in time at which the observation target in the image has a such size that can be easily recognized (visually recognized), the user can set the enlargement ratio of the image to a constant value by pressing the predetermined switch 73 of the operation unit 7. According to the configuration described above, the sizes of images in different regions of interest of the subject displayed on the display unit 16 are not changed, so that it is easy to compare the sizes of observation (diagnosis) targets in the respective images.

A distance D between the top surface (front surface) of the table 4 and the centerline O of the subject is set to, for example, about from 10 cm to 20 cm. This value may be input by the user through the input unit 17 before the start of use of the X-ray image diagnostic apparatus 1 or can be stored in advance in the storage unit 18.

This value can be changed for each subject or fixed for all subjects.

The X-ray image diagnostic apparatus of the present invention has been described above, but the present invention is not limited to the configuration of the embodiment described above. For example, the X-ray image diagnostic apparatus of the present invention can have any other arbitrary configuration added to the configuration of the embodiment described above or replaced with any configuration having the same function as the configuration of the embodiment described above.

In addition, it should be considered that the embodiment disclosed herein is an example in all respects and is not restricted. The scope of the present invention is indicated not by the description of the above embodiment but by the scope of claims, and further includes meanings equivalent to the scope of claims and all changes (modification examples) within the scope.

For example, a configuration (technology) for keeping the distance (SID) between the X-ray source and the detector or the enlargement ratio of the X-ray image of the subject constant can be applied to other X-ray image diagnostic apparatuses including an over-table tube proximity fluoroscopic table in which the positional relationship between the X-ray source and the detector with respect to the table is reversed.

In the embodiment described above, the main frame unit that holds the table 4 is supported so as to be movable up and down and rotatable with respect to the support unit 2, and the tower unit 3 is supported so as to be slidable with respect to the main frame unit. However, the present invention is not limited thereto. For example, the tower unit 3 can be supported so as to be movable up and down and rotatable with respect to the support unit 2, and the main frame unit that holds the table 4 can be supported so as to be slidable with respect to the tower unit 3.

### REFERENCE OF SIGNS

1 X-ray image diagnosis apparatus
2 Support unit
3 Tower unit
4 Table
5 X-ray tube (X-ray source)
6 Detector 6
7 Operation unit
71 Monitor
72 Operation handle
73 Switch
8 Mounting unit
9 Grid detection unit
10 Displacement mechanism
11 Tower unit moving mechanism
12 X-ray tube moving mechanism
121 Arm
13 Detector moving mechanism
131 Arm
14 Control unit
15 Image processing unit
16 Display
17 Input unit
18 Storage unit
G Grid
G1 Transmission portion
G2 Absorption portion
S Installation surface
T Distance
U1 Distance
U2 Distance
O Centerline
a, A Distance between detector 6 and centerline O
b, B Distance between X-ray tube 5 and centerline O

As used herein, a computing device broadly includes some form of an input device for receiving data, an output device for outputting data in tangible form (e.g. printing or transmitting data, or displaying on a computer screen), a memory for storing data as well as computer code, and a processor/microprocessor for executing computer code wherein said computer code resident in the memory will physically cause said processor/microprocessor to read-in data via said input device, process said data within said microprocessor and output said processed data via said output device.

It will be further understood by those of skill in the art that the apparatus and devices and the elements herein, without limitation, and including the sub components such as operational structures, circuits, communication pathways, and related elements, control elements of all kinds, display circuits and display systems and elements, any necessary driving elements, inputs, sensors, detectors, memory elements, processors and any combinations of these structures etc. as will be understood by those of skill in the art as also being identified as or capable of operating the systems and devices and subcomponents noted herein and structures that accomplish the functions without restrictive language or label requirements since those of skill in the art are well versed in related X-ray image diagnostic apparatus and imaging devices, systems, and arrangements, including related radiotherapy operational controls and technologies of radiographic devices and all their sub components, including various circuits and components and combinations of circuits and combinations of components for such devices and for all related hand held type devices, without departing from the scope and spirit of the present invention.

Although only a few embodiments have been disclosed in detail above, other embodiments are possible and the inventors intend these to be encompassed within this specification. The specification describes certain technological solutions to solve the technical problems that are described expressly and inherently in this application. This disclosure describes embodiments, and the claims are intended to cover any modification or alternative or generalization of these embodiments which might be predictable to a person having ordinary skill in the art.

Those of skill would further appreciate that the various illustrative logical blocks, modules, operating circuits, and algorithm steps described in connection with the embodiments disclosed herein may be implemented as electronic hardware, computer software running on a specific purpose machine that is programmed to carry out the operations described in this application, or combinations of both. To clearly illustrate this interchangeability of hardware and software, various illustrative components, blocks, modules, circuit illustrations, step-modes, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the exemplary embodiments.

The various illustrative logical blocks, modules, and circuits described in connection with the embodiments disclosed herein, may be implemented or performed with a general or specific purpose processor, or with hardware that carries out these functions, e.g., a Digital Signal Processor (DSP), an Application Specific Integrated Circuit (ASIC), a Field Programmable Gate Array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. The processor can be part of a computer system that also has an internal bus connecting to cards or other hardware, running based on a system BIOS or equivalent that contains startup and boot software, system memory which provides temporary storage for an operating system, drivers for the hardware and for application programs, disk interface which provides an interface between internal storage device(s) and the other hardware, an external peripheral controller which interfaces to external devices such as a backup storage device, and a network that connects to a hard wired network cable such as Ethernet or may be a wireless connection such as a RF link running under a wireless protocol such as 802.11. Likewise, an external bus may be any of but not limited to hard wired external busses such as IEEE-1394 or USB. The computer system can also have a user interface port that communicates with a user interface, and which receives commands entered by a user, and a video output that produces its output via any kind of video output format, e.g., VGA, DVI, HDMI, display port, or any other form. This may include laptop or desktop computers, and may also include portable computers, including cell phones, tablets such as the IPAD™ and Android™ platform tablet, and all other kinds of computers and computing platforms.

A processor may also be implemented as a combination of computing devices, e.g., a combination of a DSP and a microprocessor, a plurality of microprocessors, one or more microprocessors in conjunction with a DSP core, or any other such configuration. These devices may also be used to select values for devices as described herein.

The steps of a method or actions or algorithms described in connection with the embodiments disclosed herein may be embodied directly in hardware, in a software module executed by a processor, using cloud computing, or in combinations. A software module may reside in Random Access Memory (RAM), flash memory, Read Only Memory (ROM), Electrically Programmable ROM (EPROM), Electrically Erasable Programmable ROM (EEPROM), registers, hard disk, a removable disk, a CD-ROM, or any other form of tangible storage medium that stores tangible, non-transitory computer based instructions. An exemplary storage medium is coupled to the processor such that the processor can read information from, and write information to, the storage medium. In the alternative, the storage medium may be integral to the processor. The processor and the storage medium may reside in reconfigurable logic of any type.

Those of skill in the particular art will be recognized as having and having access to sophisticated radiotherapy systems, circuits, and methods such that the skill level is high in science, technology, computers, programming, circuit design, and arrangement such that the described elements herein, after and following a review of this inventive disclosure and the inventive details herein, will be understood by those of skill in the art.

In one or more exemplary embodiments, the functions described may be implemented in hardware, software, firmware, or any combination thereof. If implemented in software, the functions may be stored on or transmitted over as one or more instructions or code on a computer-readable medium. Computer-readable media includes both computer storage media and communication media including any medium that facilitates transfer of a computer program from one place to another. A storage media may be any available media that can be accessed by a computer. By way of example, and not limitation, such computer-readable media can comprise RAM, ROM, EEPROM, CD-ROM or other optical disk storage, magnetic disk storage or other magnetic storage devices, or any other medium that can be used to carry or store desired program code in the form of instructions or data structures and that can be accessed by a computer.

The memory storage can also be rotating magnetic hard disk drives, optical disk drives, or flash memory based storage drives or other such solid state, magnetic, or optical storage devices. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media. The computer readable media can be an article comprising a machine-readable non-transitory tangible medium embodying information indicative of instructions that when performed by one or more machines result in computer implemented operations comprising the actions described throughout this specification.

Operations as described herein can be carried out on or over a web site. The website can be operated on a server computer, or operated locally, e.g., by being downloaded to the client computer, or operated via a server farm. The website can be accessed over a mobile phone or a PDA, or on any other client. The website can use HTML code in any form, e.g., MHTML, or XML, and via any form such as cascading style sheets (“CSS”) or other.

The computers described herein may be any kind of computer, either general purpose, or some specific purpose computer such as a workstation. The programs may be written in C, or Java, Brew or any other programming language. The programs may be resident on a storage medium, e.g., magnetic or optical of any kind developed now or later developed e.g. the computer hard drive, a removable disk or media such as a memory stick or SD media, or other electronic recording medium. The programs may also be run locally, on a station, or over a an open or closed network without limitations thereto, for example, with a server or other machine sending signals to the local machine, which allows the local machine to carry out the operations described herein.

Also, the inventors intend that only those claims which use the words “means for” (specifically requiring the phrase “for” in “means for”) are intended to be interpreted under 35 USC 112 (f) paragraph. Moreover, no limitations from the specification are intended to be read into any claims, unless those limitations are expressly included in the claims.

It will be further understood that the method steps described herein shall be understood additionally as descriptive algorithms for the operation of the enclosed units, switches, modes, and devices and units to which they apply.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

What is claimed is:

1. An X-ray image diagnostic apparatus, comprising:
a table that has a front surface, on which a subject is placed, and a back surface;
an X-ray source that is disposed beneath said table and emits X-rays to said subject;
a detector that is disposed above said table and detects said X-rays penetrating said subject placed on said table, wherein said X-ray source and said X-ray detector are face to face with each other;
a first moving mechanism that moves said X-ray source in an approaching-and-away direction relative to the table;
a second moving mechanism that moves the detector in said approaching-and-away direction relative to said table; and
a control unit that controls a distance between said X-ray source and said detector by controlling an action of said first moving mechanism and said second moving mechanism.

2. The X-ray image diagnostic apparatus, according to claim 1, wherein:
said control unit controls said action of said second moving mechanism in response to an input from an operator and further controls said action of said first moving mechanism that moves said X-ray source interlockingly with movement of said detector controlled by said second moving mechanism.

3. The X-ray image diagnostic apparatus, according to claim 2, wherein:
said control unit controls said action of said first moving mechanism that keeps a distance (SID i.e., source image distance) between said X-ray source and said detector being constant within a movable range of said X-ray source.

4. The X-ray image diagnostic apparatus, according to claim 3, further comprising:
a mounting unit that mounts a scattered ray removal grid on the detector; and
a grid detection unit that detects mounting of said grid on said mounting unit and a kind of said grid when mounted,
wherein said control unit determines said distance based on said kind of said grid detected by said grid detection unit.

5. The X-ray image diagnostic apparatus, according to claim 2, wherein:
said control unit controls said action of said first moving mechanism so that an enlargement ratio of an X-ray image of said subject is being constant.

6. The X-ray image diagnostic apparatus, according to claim 1, further comprising:
a lifting mechanism that moves up-and-down said first moving mechanism together with said second moving mechanism when said table is horizontal,
wherein said control unit controls said action of said first moving mechanism so that said X-ray source moves interlockingly with said table that moves up-and-down using said lifting mechanism when said table is horizontal.

7. The X-ray image diagnostic apparatus, according to claim 6, wherein:
said control unit controls said action of said first moving mechanism so that said X-ray source moves in an approaching direction to said table when a distance between said installation surface on which said X-ray image diagnostic apparatus is placed and said X-ray source is a predetermined value.
